(19) United States
(12) Patent Application Publication
(10) Pub. No.: US 2022/0237524 A1
(43) Pub. Date: Jul. 28, 2022

(54) LEARNING MODEL GENERATION METHOD, PROGRAM, STORAGE MEDIUM, AND LEARNED MODEL

(71) Applicant: DAIKIN INDUSTRIES, LTD., Osaka-shi, Osaka (JP)

(72) Inventors: Rumi KAWABE, Osaka-shi, Osaka (JP); Tatsuya TAKAKUWA, Osaka-shi, Osaka (JP); Haruhisa MASUDA, Osaka-shi, Osaka (JP); Kei KURAMOTO, Osaka-shi, Osaka (JP)

(73) Assignee: DAIKIN INDUSTRIES, LTD., Osaka-shi, Osaka (JP)

(21) Appl. No.: 17/610,085

(22) PCT Filed: May 12, 2020
(86) PCT No.: PCT/JP2020/018967

§ 371 Date: Nov. 9, 2021

(30) Foreign Application Priority Data
May 16, 2019 (JP) 2019-092818

(51) Int. Cl. G06N 20/20; G16C 20/70

(52) U.S. Cl. CPC G06N 20/20; G16C 20/70

(57) ABSTRACT

A learning model generation method may include obtaining, by a processor, as teacher data, information including at least first base material information regarding a first base material, first treatment agent information regarding a first surface-treating agent, and a first evaluation of a first article; learning, by the processor, based on the teacher data; and generating, by the processor, a learning model based on the learning. A second article may be obtained by fixing a second surface-treating agent onto a second base material. The learning model may be configured to receive input information, which is different from the teacher data, as an input, and output a second evaluation of the second article. The input information may include at least second base material information regarding the second base material, and second treatment agent information regarding the second surface-treating agent.

CROSS-REFERENCE TO RELATED APPLICATION(S)

This application is a § 371 of International Application No. PCT/JP2020/018967, filed on May 12, 2020, claiming priority from Japanese Patent Application No. 2019-092818, filed on May 16, 2019, the disclosures of which are incorporated by reference herein in their entireties.

### 1. Field

The present disclosure relates to a learning model generation method, a program, a storage medium storing the program, and a learned model.

### 2. Description of Related Art

Patent Literature 1 (JPA No. 2018-535281) discloses a preferable combination of water-repellent agents.

Patent Literature 2 (JPB No. 4393595) discloses an optimization analysis device and a storage medium storing an optimization analysis program.

### SUMMARY

Discovery of a preferable combination of water-repellent agents, and the like, might require tests, evaluations, and the like, to be conducted repeatedly, resulting in a heavy burden in terms of time and cost.

A learning model generation method according to a first aspect generates a learning model for determining by using a computer an evaluation of an article in which a surface-treating agent is fixed onto a base material. The learning model generation method includes an obtaining operation, a learning operation, and a generating operation. In the obtaining operation, the computer obtains teacher data. The teacher data includes base material information, treatment agent information, and the evaluation of the article. The base material information is information regarding a base material. The treatment agent information is information regarding the surface-treating agent. In the learning operation, the computer learns on the basis of a plurality of the teacher data obtained in the obtaining operation. In the generating operation, the computer generates the learning model on the basis of a result of learning in the learning operation. The article is obtained by fixing the surface-treating agent onto the base material. The learning model receives input information as an input, and outputs the evaluation. The input information is unknown information different from the teacher data. The input information includes at least the base material information and the treatment agent information.

The learning model thus generated enables evaluation by using a computer, and in turn reduction of extensive time and cost required for conducting the evaluation.

A learning model generation method according to a second aspect includes an obtaining operation, a learning operation, and a generating operation. In the obtaining operation, a computer obtains teacher data. The teacher data includes base material information, treatment agent information, and an evaluation. The base material information is information regarding a base material. The treatment agent information is information regarding a surface-treating agent. The evaluation is regarding an article in which the surface-treating agent is fixed onto the base material. In the learning operation, the computer learns on the basis of a plurality of the teacher data obtained in the obtaining operation. In the generating operation, the computer generates the learning model on the basis of a result of learning in the learning operation. The article is obtained by fixing the surface-treating agent onto the base material. The learning model receives input information as an input, and outputs the evaluation. The input information is unknown information different from the teacher data. The input information includes at least the base material information and information regarding the evaluation.

A learning model generation method according to a third aspect is the learning model generation method according to the first aspect or the second aspect, in which in the learning operation, the learning is performed by a regression analysis and/or ensemble learning that is a combination of a plurality of regression analyses.

A program according to a fourth aspect is a program with which a computer determines, by using a learning model, an evaluation of a base material onto which a surface-treating agent is fixed. The program includes an input operation, a determination operation, and an output operation. In the input operation, the computer receives input information as an input. In the determination operation, the computer determines the evaluation. In the output operation, the computer outputs the evaluation determined in the determination operation. The article is obtained by fixing the surface-treating agent onto the base material. The learning model learns, as teacher data, base material information, which is information regarding the base material, treatment agent information, which is information regarding the surface-treating agent to be fixed onto the base material, and the evaluation. The input information is unknown information different from the teacher data, including the base material information and the treatment agent information.

A program according to a fifth aspect is a program with which a computer determines, by using a learning model, treatment agent information that is optimal (or improved) for fixation onto a base material. The program includes an input operation, a determination operation, and an output operation. In the input operation, the computer receives input information as an input. In the determination operation, the computer determines the treatment agent information that is optimal (or improved). In the output operation, the computer outputs the treatment agent information that is optimal (or improved) determined in the determination operation. The learning model learns, as teacher data, base material information, treatment agent information, and an evaluation. The base material information is information regarding a base material. The treatment agent information is information regarding a surface-treating agent. The evaluation is regarding an article in which the surface-treating agent is fixed onto the base material. The treatment agent information is information regarding a surface-treating agent to be fixed onto the base material. The input information is unknown information different from the teacher data. The input information includes at least the base material information and information regarding the evaluation. The article is obtained by fixing the surface-treating agent onto the base material.

A program according to a sixth aspect is the program according to the fourth aspect or the fifth aspect, in which the evaluation is any of water-repellency information, oil-repellency information, antifouling property information, or processing stability information. The water-repellency information is information regarding water-repellency of the article. The oil-repellency information is information regarding oil-repellency of the article. The antifouling property information is information regarding an antifouling property of the article. The processing stability information is information regarding processing stability of the article.

A program according to a seventh aspect is the program according to any of the fourth aspect to the sixth aspect, in which the base material is a textile product.

A program according to an eighth aspect is the program according to the seventh aspect, in which the base material information includes information regarding at least a type of the textile product and a type of a dye. The treatment agent information includes information regarding at least a type of a monomer constituting a repellent polymer contained in the surface-treating agent, a content of a monomeric unit in the polymer, a content of the repellent polymer in the surface-treating agent, a type of a solvent and a content of the solvent in the surface-treating agent, and a type of a surfactant and a content of the surfactant in the surface-treating agent.

A program according to a ninth aspect is the program according to the eighth aspect, in which the teacher data includes environment information during processing of the base material. The environment information includes information regarding any of temperature, humidity, curing temperature, or processing speed during the processing of the base material. The base material information further includes information regarding any of a color, a weave, basis weight, yarn thickness, or zeta potential of the textile product. The treatment agent information further includes information regarding any item of: a type and a content of an additive to be added to the surface-treating agent; pH of the surface-treating agent; or zeta potential thereof.

A program according to a tenth aspect is a storage medium storing the program according to any of the fourth aspect to the ninth aspect.

A learned model according to an eleventh aspect is a learned model for causing a computer to function. The learned model performs calculation based on a weighting coefficient of a neural network with respect to base material information and treatment agent information being input to an input layer of the neural network. The learned model outputs water-repellency information or oil-repellency information of a base material from an output layer of the neural network on the basis of a result of the calculation. The base material information is information regarding the base material. The treatment agent information is information regarding a surface-treating agent. The weighting coefficient is obtained through learning of at least the base material information, the treatment agent information, and an evaluation as teacher data. The evaluation is regarding the article in which the surface-treating agent is fixed onto the base material. The article is obtained by fixing the surface-treating agent onto the base material.

A learned model according to a twelfth aspect is a learned model for causing a computer to function. The learned model performs calculation based on a weighting coefficient of a neural network with respect to base material information and information regarding an evaluation being input to an input layer of the neural network. The learned model outputs treatment agent information that is optimal (or improved) for a base material from an output layer of the neural network on the basis of a result of the calculation. The base material information is information regarding the base material. The weighting coefficient is obtained through learning of at least the base material information, the treatment agent information, and the evaluation as teacher data. The treatment agent information is information regarding a surface-treating agent to be fixed onto the base material. The evaluation is regarding an article in which the surface-treating agent is fixed onto the base material. The article is obtained by fixing the surface-treating agent onto the base material.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 shows a configuration of a learning model generation device;

FIG. 2 shows a configuration of a user device;

FIG. 3 shows an example of a decision tree;

FIG. 4 shows an example of a feature space divided by the decision tree;

FIG. 5 shows an example of a support vector machine (SVM);

FIG. 6 shows an example of a feature space;

FIG. 7 shows an example of a neuron model in a neural network;

FIG. 8 shows an example of a neural network;

FIG. 9 shows an example of teacher data;

FIG. 10 is a flow chart of an operation of the learning model generation device; and

FIG. 11 is a flow chart of an operation of the user device.

### DETAILED DESCRIPTION

A learning model according to an embodiment of the present disclosure is described hereinafter. Note that the embodiment described below is a specific example which does not limit the technical scope of the present disclosure, and may be modified as appropriate without departing from the spirit of the present disclosure.

### (1) Summary

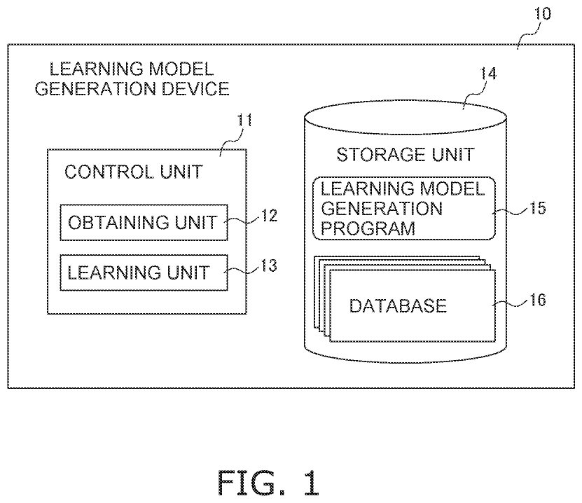

FIG. 1 is a diagram showing a configuration of a learning model generation device. FIG. 1 is a diagram showing a configuration of a user device.

The learning model is generated by a learning model generation device 10, which is at least one computer, that is configured to obtain and learn using teacher data. The learning model thus generated is, as a learned model: implemented to a general-purpose computer or terminal; downloaded as a program, or the like; or distributed in a state of being stored in a storage medium, and is used in a user device 20, which is at least one computer.

The learning model is configured to output a correct answer for unknown information that is different from the teacher data. Furthermore, the learning model can be updated so as to output a correct answer for various types of data that is input.

### (2) Configuration of Learning Model Generation Device 10

The learning model generation device 10 generates a learning model to be used in the user device 20 described later.

The learning model generation device 10 is a device having a function of a computer. Alternatively, the learning model generation device 10 may include a communication interface such as a network interface card (NIC) and a direct memory access (DMA) controller, and is configured to communicate with the user device 20, and the like, through a network. Although the learning model generation device 10 is illustrated in FIG. 1 as a single device, the learning model generation device 10 may be a cloud server or a group of cloud servers implemented in a cloud computing environment. Consequently, in terms of a hardware configuration, the learning model generation device 10 is not required to be accommodated in a single housing or be provided as a single device. For example, the learning model generation device 10 is configured in such a way that hardware resources thereof are dynamically connected and disconnected according to a load.

The learning model generation device 10 includes a control unit 11 and a storage unit 14.

### (2-1) Control Unit 11

The control unit 11 is, for example, a central processing unit (CPU) and controls an overall operation of the learning model generation device 10. The control unit 11 causes each of the function units described below to function appropriately, and executes a learning model generation program 15 stored in advance in the storage unit 14. The control unit 11 includes the function units such as an obtaining unit 12, and a learning unit 13.

In the control unit 11, the obtaining unit 12 obtains teacher data that is input to the learning model generation device 10, and stores the teacher data thus obtained in a database 16 built in the storage unit 14. The teacher data may be either directly input to the learning model generation device 10 by a user of the learning model generation device 10, or obtained from another device, or the like, through a network. A manner in which the obtaining unit 12 obtains the teacher data is not limited. The teacher data is information for generating a learning model configured to achieve a learning objective. As used herein, the learning objective is any of: outputting an evaluation of an article in which a surface-treating agent is fixed onto a base material; or outputting treatment agent information that is optimal (or improved) for fixation onto the base material. Details thereof are described later.

The learning unit 13 extracts a learning dataset from the teacher data stored in the storage unit 14, to automatically perform machine learning. The learning dataset is a set of data, whose correct answer to an input is known. The learning dataset to be extracted from the teacher data is different depending on the learning objective. The learning by the learning unit 13 generates the learning model.

### (2-2) Machine Learning

An approach of the machine learning performed by the learning unit 13 is not limited as long as the approach is supervised learning that employs the learning dataset. A model or an algorithm used for the supervised learning is exemplified by regression analysis, a decision tree, SVM, neural network, ensemble learning, random forest, and the like.

Examples of the regression analysis include linear regression analysis, multiple regression analysis, and logistic regression analysis. The regression analysis is an approach of applying a model between input data (e.g., an explanatory variable) and learning data (e.g., an objective variable) through the least-squares method, or the like. The dimension of the explanatory variable is one in the linear regression analysis, and two in the multiple regression analysis. The logistic regression analysis uses a logistic function (e.g., a sigmoid function) as the model.

The decision tree is a model for combining a plurality of classifiers to generate a complex classification boundary. The decision tree is described later in detail.

The SVM is an algorithm of generating a two-class linear discriminant function. The SVM is described later in detail.

The neural network is modeled from a network formed by connecting neurons in the human nervous system with synapses. The neural network, in a narrow sense, refers to a multi-layer perceptron using backpropagation. The neural network is typically exemplified by a convolutional neural network (CNN) and a recurrent neural network (RNN). The CNN is a type of feedforward neural network which is not fully connected (e.g., is sparsely connected). The neural network is described later in detail.

The ensemble learning is an approach of improving classification performance through combination of a plurality of models. An approach used for the ensemble learning is exemplified by bagging, boosting, and random forest. Bagging is an approach of causing a plurality of models to learn by using bootstrap samples of the learning data, and determining an evaluation of new input data by majority vote of the plurality of models. Boosting is an approach of weighting learning data depending on learning results of bagging, and learning incorrectly classified learning data more intensively than correctly classified learning data. Random forest is an approach of, in the case of using a decision tree as a model, generating a set of decision trees (e.g., a random forest) constituted of a plurality of weakly correlated decision trees. Random forest is described later in detail.

### (2-2-1) Decision Tree

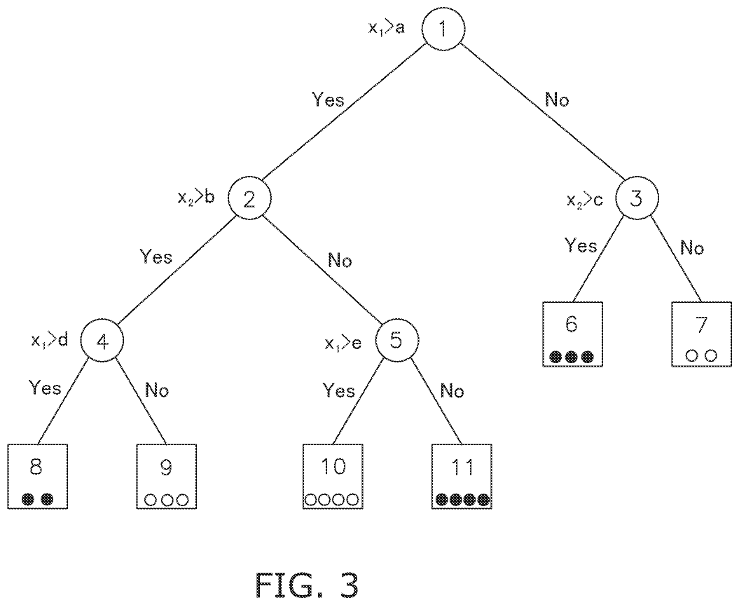

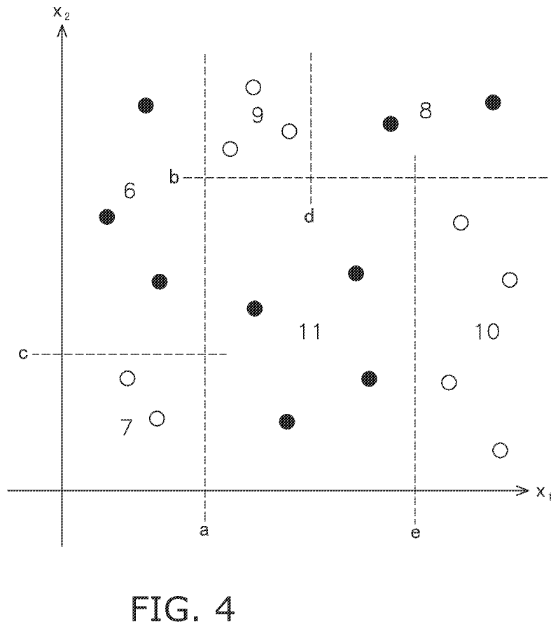

The decision tree is a model for combining a plurality of classifiers to obtain a complex classification boundary (e.g., a non-linear discriminant function, and the like). A classifier is, for example, a rule regarding a magnitude relationship between a value on a specific feature axis and a threshold value. A method for constructing a decision tree from learning data is exemplified by the divide-and-conquer method of repetitively obtaining a rule (e.g., a classifier) for dividing a feature space into two. FIG. 3 shows an example of a decision tree constructed by the divide-and-conquer method. FIG. 4 shows a feature space divided by the decision tree of FIG. 3. In FIG. 4, learning data is indicated by a white dot or a black dot, and each learning data is classified by the decision tree of FIG. 3 into a class of white dot or a class of black dot. FIG. 3 shows nodes numbered from 1 to 11, and links labeled “Yes” or “No” connecting the nodes. In FIG. 3, terminal nodes (e.g., leaf nodes) are indicated by squares, while non-terminal nodes (e.g., root nodes and intermediate nodes) are indicated by circles. The terminal nodes are those numbered from 6 to 11, while the non-terminal nodes are those numbered from 1 to 5. White dots or black dots representing the learning data are shown in each of the terminal nodes. A classifier is provided to each of the non-terminal nodes. The classifiers are rules for determining magnitude relationships between values on feature axis x₁, x₂ and threshold values a to e. Labels provided to links show determination results of the classifiers. In FIG. 4, the classifiers are shown by dotted lines, and regions divided by the classifiers are each provided with the number of the corresponding node.

In the process of constructing an appropriate decision tree by the divide-and-conquer method, consideration of the following three elements (a) to (c) may be required.

(a) Selection of feature axis and threshold values for constructing classifiers.

(b) Determination of terminal nodes. For example, the number of classes to which learning data contained in one terminal node belongs. Alternatively, a choice of how much a decision tree is to be pruned (how many identical subtrees are to be given to a root node).

(c) Assignment of a class to a terminal node by majority vote.

For example, CART, ID3, and C4.5 are used for learning of a decision tree. CART is an approach of generating a binary tree as a decision tree by dividing a feature space into two at each node except for terminal nodes for each feature axis, as shown in FIG. 3 and FIG. 4.

In the case of learning using a decision tree, it is important to divide a feature space at an optimal candidate division point at a non-terminal node, in order to improve classification performance of learning data. A parameter for evaluating a candidate division point of a feature space may be an evaluation function referred to as impurity. Function I(t) representing impurity of a node t is exemplified by parameters represented by following equations (1-1) to (1-3). K represents the number of classes.

$$\begin{matrix}[ Expression 1 ] & \\ ( a ) Error rate at node t & \end{matrix}$$

$$I ( t ) = 1 - {max}_{i} P ( {C}_{i} \vert t ) \qquad ( 1 - 1 )$$

$$\text{(b) Cross entropy (degree of deviation)}$$

$$I ( t ) = - {\sum }_{i = 1}^{K} P ( {C}_{i} \vert t ) ln P ( {C}_{i} \vert t ) \qquad ( 1 - 2 )$$

$$\text{(c) Gini coefficient}$$

$$I ( t ) = {\sum }_{i = 1}^{K} {\sum }_{j \neq i} P ( {C}_{i} \vert t ) P ( {C}_{j} \vert t ) = {\sum }_{i = 1}^{K} P ( {C}_{i} \vert t ) ( 1 - P ( {C}_{i} \vert t ) ) \qquad ( 1 - 3 )$$

In the above equations, a probability P(Cᵢ|t) represents a posterior probability of a class Cᵢ at the node t, i.e., a probability of data in the class Cᵢ being chosen at the node t. The probability P(Cⱼ|t) in the second member of the equation (1-3) refers to a probability of data in the class Cᵢ being erroneously taken as a j-th (≠i-th) class, and thus the second member of the equation represents an error rate at the node t. The third member of the equation (1-3) represents a sum of variances of the probability P(Cᵢ|t) regarding all classes.

In the case of dividing a node with the impurity as an evaluation function, for example, an approach of pruning a decision tree to fall within an allowable range defined by an error rate at the node and complexity of a decision tree.

### (2-2-2) SVM

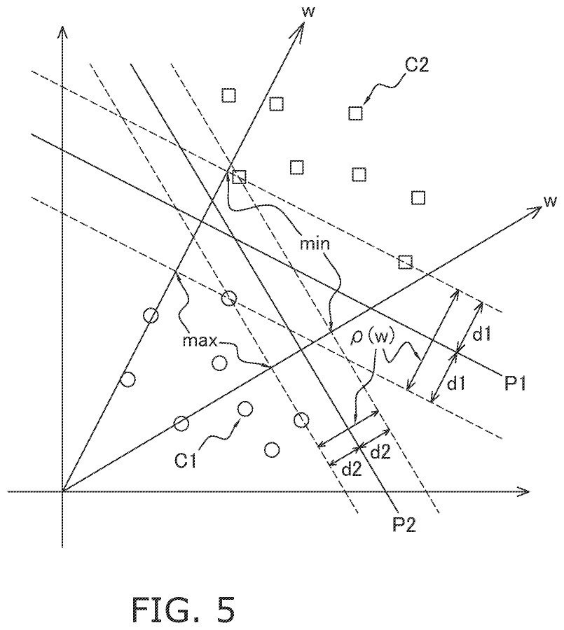

The SVM is an algorithm of obtaining a two-class linear discriminant function achieving the maximum margin. FIG. 5 illustrates the SVM. The two-class linear discriminant function refers to, in the feature space shown in FIG. 5, classification hyperplanes P1 and P2, which are hyperplanes for linear separation of learning data of two classes C1 and C2. In FIG. 5, learning data of the class C1 is indicated by circles, while the learning data of the class C2 is indicated by squares. A margin of a classification hyperplane refers to a distance between the classification hyperplane and learning data closest to the classification hyperplane. FIG. 5 shows a margin d1 of the classification hyperplane P1 and a margin d2 of the classification hyperplane P2. The SVM obtains an optimal classification hyperplane P1, which is a classification hyperplane having the maximum margin. The minimum value d1 of a distance between learning data of one class C1 and the optimal classification hyperplane P1 is equal to the minimum value d1 of a distance between learning data of the other class C2 and an optimal classification hyperplane P2.

The following equation (2-1) represents a learning dataset DL used for the supervised learning of a two-class problem shown in FIG. 5.

[Expression 2]

D_{L}={(tᵢ, xᵢ)}(i=1, . . . , N) (2-1)

The learning dataset D_{L} is a set of pairs of learning data (e.g., a feature vector) x₁ and teacher data tᵢ={−1, +1}. N represents the number of elements in the learning dataset D_{L}. The teacher data tᵢ indicates to which one of the classes C1 and C2 the learning data xᵢ belongs. The class C1 is a class of tᵢ=−1, while the class C2 is a class of tᵢ=+1.

A normalized linear discriminant function which holds for all pieces of the learning data xᵢ in FIG. 5 is represented by the following two equations (2-2) and (2-3). A coefficient vector is represented by w, while a bias is represented by b.

[Expression 3]

In the case of tᵢ=+1 w^{T}xᵢ+b≥+1 (2-2)

In the case of tᵢ=−1 w^{T}xᵢ+b≤−1 (2-3)

The two equations are represented by the following equation (2-4).

[Expression 4]

tᵢ(w^{T}xᵢ+b)≥1 (2-4)

In a case in which the classification hyperplanes P1 and P2 are represented by the following equation (2-5), a margin d thereof is represented by the equation (2-6).

$$[ Expression 5 ]$$

$${w}^{T} x + b = 0 \qquad ( 2 - 5 )$$

$$d = \frac{1}{2} ρ ( w ) = \frac{1}{2} ( {min}_{{x}_{i} ∈ {C}_{2}} \frac{{w}^{T} {x}_{i}}{‖ w \vert \vert} - {max}_{{x}_{i} ∈ {C}_{1}} \frac{{w}^{T} {x}_{i}}{‖ w \vert \vert} ) \qquad ( 2 - 6 )$$

In the equation (2-6), p(w) represents a minimum value of a difference in length of projection of the learning data xᵢ of the classes C1 and C2, on a normal vector w of each of the classification hyperplanes P1 and P2. The terms “min” and “max” in the equation (2-6) represent respective points denoted by symbols “min” and “max” in FIG. 5. In FIG. 5, the optimal classification hyperplane is the classification hyperplane P1 of which margin d is the maximum.

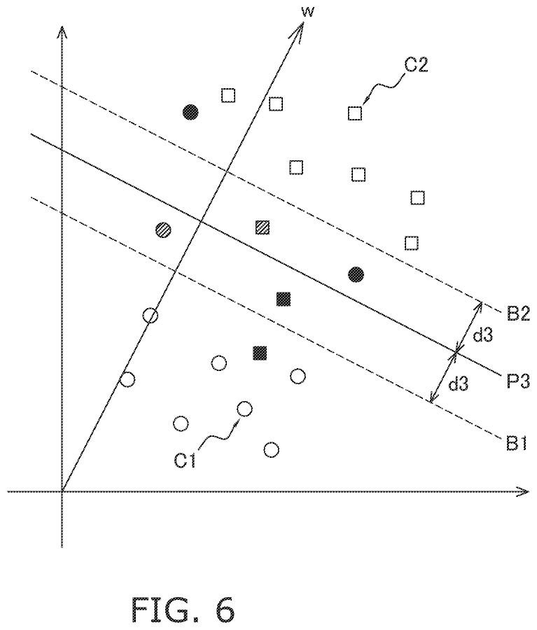

FIG. 5 shows a feature space in which linear separation of learning data of the two classes is possible. FIG. 6 shows a feature space similar to that of FIG. 5, in which linear separation of learning data of the two classes is not possible. In the case in which linear separation of learning data of the two classes is not possible, the following equation (2-7) obtained by expanding the equation (2-4) by introducing a slack variable ξᵢ can be used.

[Expression 6]

tᵢ(w^{T}xᵢ+b)−1+ξᵢ≥0 (2-7)

The slack variable ξᵢ is used only during learning and has a value of at least 0. FIG. 6 shows a classification hyperplane P3, margin boundaries B1 and B2, and a margin d3. An equation for the classification hyperplane P3 is identical to the equation (2-5). The margin boundaries B1 and B2 are hyperplanes spaced apart from the classification hyperplane P3 by the margin d3.

When the slack variable ξᵢ is 0, the equation (2-7) is equivalent to the equation (2-4). In this case, as indicated by open circles or open squares in FIG. 6, the learning data xᵢ satisfying the equation (2-7) is correctly classified within the margin d3. In this case, a distance between the learning data xᵢ and the classification hyperplane P3 is greater than the margin d3.

When the slack variable ξᵢ is greater than 0 and no greater than 1, as indicated by a hatched circle or a hatched square in FIG. 6, the learning data xᵢ satisfying the equation (2-7) is correctly classified, beyond the margin boundaries B1 and B2, and not beyond the classification hyperplane P3. In this case, a distance between the learning data xᵢ and the classification hyperplane P3 is less than the margin d3.

When the slack variable ξᵢ is greater than 1, as indicated by filled circles or filled squares in FIG. 6, the learning data xᵢ satisfying the equation (2-7) is beyond the classification hyperplane P3 and incorrectly classified.

By thus using the equation (2-7) to which the slack variable ξᵢ is introduced, the learning data xᵢ can be classified even in the case in which linear separation of the learning data of two classes is not possible.

As described above, a sum of the slack variables ξᵢ of all pieces of the learning data xᵢ represents the upper limit of the number of pieces of the learning data xᵢ incorrectly classified. Here, an evaluation function Lₚ is defined by the following equation (2-8).

[Expression 7]

Lₚ(w,ξ)=½w^{T}w+CΣᵢ₌₁^{N}ξᵢ (2-8)

A solution (w,ξ) that minimizes an output value of the evaluation function Lₚ is to be obtained. In the equation (2-8), a parameter C in the second expression represents strength of a penalty for incorrect classification. The greater parameter C might require a solution further prioritizing reduction of the number of incorrect classifications (second expression) over reduction of the norm of w (first expression).

### (2-2-3) Neural Network

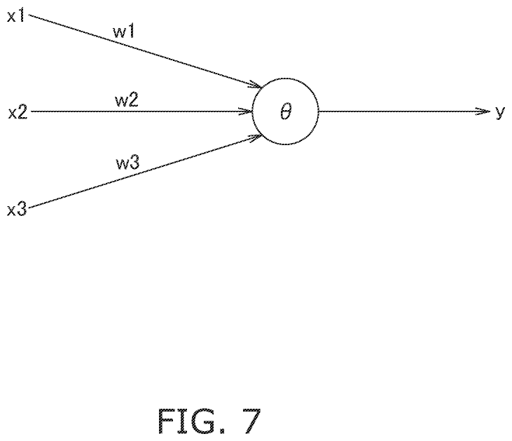

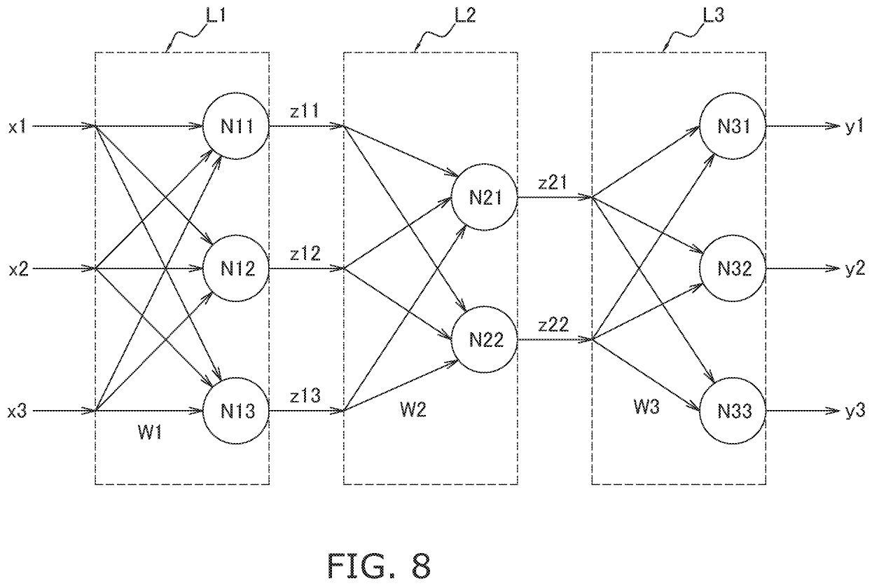

FIG. 7 is a schematic view of a model of a neuron in a neural network. FIG. 8 is a schematic view of a three-layer neural network constituted by combining the neuron shown in FIG. 7. As shown in FIG. 7, the neuron outputs an output y for a plurality of inputs x (inputs x1, x2, and x3 in FIG. 7). Each of the inputs x (inputs x1, x2 and x3 in FIG. 7) is multiplied by a corresponding weight w (weight w1, w2 and w3 in FIG. 7). The neuron outputs the output y by means of the following equation (3-1).

[Expression 8]

y=φ(Σᵢ₌₁ⁿxᵢwᵢ−θ) (3-1)

In the equation (3-1), the input x, the output y and the weight w are all vectors; θ is a bias; and φ denotes an activation function. The activation function is a non-linear function such as, for example, a step function (e.g., a formal neuron), a simple perceptron, a sigmoid function, or a rectified linear unit (ReLU) (e.g., a ramp function).

The three-layer neural network shown in FIG. 8 receives a plurality of input vectors x (input vectors x1, x2 and x3 in FIG. 8) from an input side (left side of FIG. 8), and outputs a plurality of output vectors y (output vectors y1, y2, and y3 in FIG. 8) from an output side (right side of FIG. 8). This neural network is constituted of three layers L1, L2, and L3.

In the first layer L1, the input vectors x1, x2, and x3 are multiplied by respective weights, and input to each of three neurons N11, N12, and N13. In FIG. 8, W1 collectively denotes the weights. The neurons N11, N12, and N13 output feature vectors z11, z12, and z13, respectively. In the second layer L2, the feature vectors z11, z12, and z13 are multiplied by respective weights, and input to each of two neurons N21 and N22. In FIG. 8, W2 collectively denotes the weights. The neurons N21 and N22 output feature vectors z21 and z22 respectively.

In the third layer L3, the feature vectors z21 and z22 are multiplied by respective weights, and input to each of three neurons N31, N32, and N33. In FIG. 8, W3 collectively denotes the weights. The neurons N31, N32, and N33 output output vectors y1, y2, and y3, respectively.

The neural network functions in a learning mode and a prediction mode. The neural network in the learning mode learns the weights W1, W2, and W3 using a learning dataset. The neural network in the prediction mode predicts classification ,and the like, using parameters of the weights W1, W2, and W3 thus learned.

Learning of the weights W1, W2, and W3 can be achieved by, for example, backpropagation. In this case, information regarding an error is propagated from the output side toward the input side such as, in other words, from a right side toward a left side of FIG. 8. The backpropagation learns the weights W1, W2, and W3 with adjustment to reduce a difference between the output y in the case in which the input x is input and the proper output y (e.g., teacher data) in each neuron.

The neural network may be configured to have more than three layers. An approach of machine learning with a neural network having four or more layers is known as deep learning.

### (2-2-4) Random Forest

Random forest is a type of the ensemble learning, and reinforces classification performance through a combination of a plurality of decision trees. The learning employing random forest generates a set constituted of a plurality of weakly correlated decision trees (e.g., a random forest). The following algorithm generates and classifies the random forest:

(A) Repeat the following from m=1 to m=M.

(a) Generate m bootstrap sample(s) Zₘ from N pieces of d-dimensional learning data.

(b) Generate m decision tree(s) by dividing each node t as follows, with Zₘ as learning data:
(i) Randomly select d′ features from d features (d′<d).
(ii) Determine a feature and a division point (threshold value) achieving the optimal division of the learning data from among the d′ features thus selected.
(iii) Divide the node t into two at the division point thus determined.

(B) Output a random forest constituted of m decision tree(s).

(C) Obtain a classification result of each decision tree in the random forest for input data. Majority vote for the classification result of each decision tree determines the classification result of the random forest.

The learning employing random forest enables weakening of correlation between decision trees, through random selection of a preset number of features used for classification at each non-terminal node of the decision tree.

### (2-3) Storage Unit 14

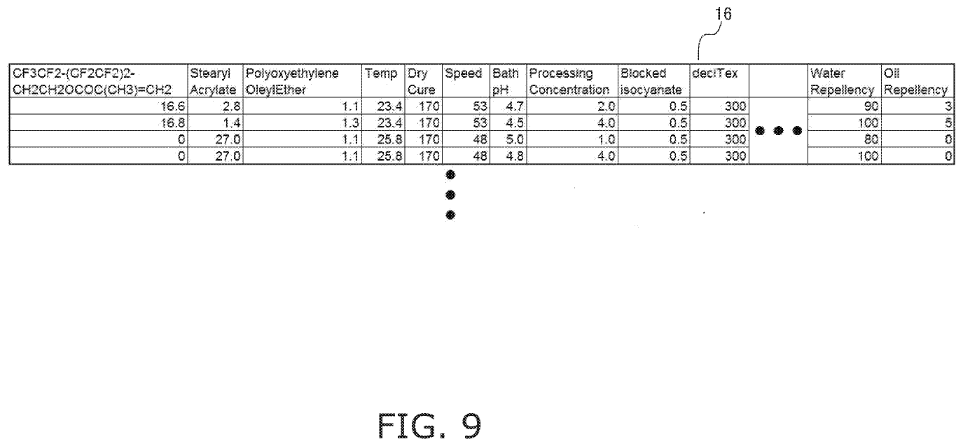

The storage unit 14 shown in FIG. 1 is an example of a non-transitory computer-readable storage medium and may be, for example, a flash memory, a random access memory (RAM), a hard disk drive (HDD), or the like. The storage unit 14 includes the learning model generation program 15 to be executed by the control unit 11, being stored in advance. The storage unit 14 is provided with the database 16 being built in, in which a plurality of the teacher data obtained by the obtaining unit 12 are stored and appropriately managed. The database 16 stores the plurality of the teacher data as shown in FIG. 9, for example. Note that FIG. 9 illustrates a part of the teacher data stored in the database 16. The storage unit 14 may also store information for generating a learning model, such as the learning dataset and test data, in addition to the teacher data.

### (3) Teacher Data

It has been found that the base material information, the treatment agent information, and the evaluation are correlated to each other.

Given this, the teacher data to be obtained for generating the learning model includes at least the base material information, the treatment agent information, and information regarding the evaluation as described below. In light of improving accuracy of an output value, the teacher data preferably further includes environment information. Note that, as a matter of course, the teacher data may also include information other than the following. The database 16 in the storage unit 14 according to the present disclosure stores a plurality of the teacher data including the following information.

### (3-1) Base Material Information

The base material information is information regarding the base material onto which the surface-treating agent is fixed.

The base material may be a textile product. The textile product includes: a fiber; a yarn; a fabric such as a woven fabric, a knitted fabric, and a nonwoven fabric; a carpet; leather; paper; and the like. In the case described hereinafter, the base material is the textile product.

Note that the learning model generated in the present embodiment may be used for the base material other than the textile product.

The base material information includes: a type of the textile product; a type of a dye with which a surface of the textile product is dyed; a thickness of fiber used for the textile product; a weave of the fiber; a basis weight of the fiber; a color of the textile product; a zeta potential of the surface of the textile product; and the like.

The base material information includes at least information regarding the type of the textile product and/or the color of the textile product, and may further include information regarding the thickness of the fiber.

Note that the teacher data shown in FIG. 9 includes the aforementioned items, which are not illustrated, as the base material information.

### (3-2) Treatment Agent Information

The treatment agent information is information regarding a surface-treating agent to be fixed onto the base material. The surface-treating agent is exemplified by a repellent agent to be fixed onto the base material for imparting water-repellency or oil-repellency thereto. In the case described hereinafter, the surface-treating agent is the repellent agent.

In the present disclosure, the repellent agent preferably contains a repellent polymer, a solvent, and a surfactant.

The repellent polymer is selected from fluorine-containing repellent polymers or non-fluorine repellent polymers. The fluorine-containing repellent polymers and the non-fluorine repellent polymers are preferably acrylic polymers, silicone polymers, or urethane polymers. The fluorine-containing acrylic polymers may contain a repeating unit derived from a fluorine-containing monomer represented by the formula CH2═C(—X)—C(═O)—Y—Z—Rf, wherein X represents a hydrogen atom, a monovalent organic group, or a halogen atom; Y represents —O— or —NH—; Z represents a direct bond or a divalent organic group; and Rf represents a fluoroalkyl group having 1 to 6 carbon atoms. The non-fluorine repellent polymers are preferably non-fluorine acrylic polymers containing a repeating unit derived from a long-chain (meth)acrylate ester monomer represented by formula (1) CH2═CA11—C(═O)—O—A12, wherein A11 represents a hydrogen atom or a methyl group; and A12 represents a linear or branched aliphatic hydrocarbon group having 10 to 40 carbon atoms.

The solvent is exemplified by water, a non-water solvent, and the like.

The surfactant is exemplified by a nonionic surfactant, a cationic surfactant, an anion surfactant, an amphoteric surfactant, and the like.

The repellent agent may also include an additive, in addition to the aforementioned components. A type of the additive is exemplified by a cross-linking agent (e.g., blocked isocyanate), an insect repellent, an antibacterial agent, a softening agent, an antifungal agent, a flame retarder, an antistatic agent, an antifoaming agent, a coating material fixative, a penetrating agent, an organic solvent, a catalyst, a pH adjusting agent, a wrinkle-resistant agent, and the like.

The treatment agent information includes a type of a monomer constituting a repellent polymer contained in the surface-treating agent, a content of the monomer in the repellent polymer, a content of the repellent polymer in the surface-treating agent, a type of a solvent and a content of the solvent in the surface-treating agent, and a type of a surfactant and a content of the surfactant in the surface-treating agent.

The treatment agent information preferably includes at least a type of a monomer constituting a repellent polymer contained in the surface-treating agent, and a content of a monomeric unit in the repellent polymer.

The treatment agent information more preferably further includes, in addition to the foregoing, a content of the repellent polymer in the surface-treating agent, a type of a solvent, and a content of the solvent in the surface-treating agent. The treatment agent information may further include, in addition to the foregoing, a type of a surfactant and a content of the surfactant in the surface-treating agent.

The treatment agent information may also include information other than the foregoing, such as information regarding a type and a content of an additive to be added to the repellent agent, a pH of the repellent agent, a zeta potential of the repellent agent; and the like. As a matter of course, the treatment agent information may include information other than the foregoing. Note that the teacher data shown in FIG. 9 includes the aforementioned items, as the treatment agent information.

### (3-3) Evaluation

The evaluation is information regarding the article in which the surface-treating agent is fixed.

The evaluation includes information regarding chemical properties such as water-repellency information, oil-repellency information, antifouling property information, processing stability information; and the like. The evaluation may include at least the water-repellency information and the oil-repellency information. The water-repellency information is information regarding water-repellency of the article after fixation of the surface-treating agent. The water-repellency information is, for example, a value of water-repellency evaluated according to JIS L1092 (spray test). The oil-repellency information is information regarding oil-repellency of the article after fixation of the surface-treating agent. The oil-repellency information is, for example, a value of oil-repellency evaluated according to AATCC 118 or ISO 14419. The antifouling property information is information regarding antifouling property of the article after fixation of the surface-treating agent. The antifouling property information is, for example, a value of antifouling property evaluated according to JIS L1919. The processing stability information is information regarding effects borne by the article and the surface-treating agent, during an operation of processing the article after fixation of the surface-treating agent. The processing stability information may have a standard each being defined according to the processing operation. For example, the processing stability is indicated by a value obtained by quantifying a degree of adhesion of a resin to a roller that applies pressure to squeeze the textile product.

Note that the teacher data shown in FIG. 9 includes as the evaluation at least one of the aforementioned items.

### (3-4) Environment Information

The environment information is regarding an environment in which the surface-treating agent is fixed onto the base material. Specifically, the environment information is information regarding, for example, a concentration of the surface-treating agent in a treatment tank, an environment of a factory, or the like, for performing processing of fixing the surface-treating agent onto the base material, or information regarding operations of processing.

The environment information may also include, for example, information regarding a temperature, a humidity, a curing temperature, a processing speed, and the like, during the processing of the base material. The environment information includes at least information regarding the concentration of the surface-treating agent in a treatment tank. Note that the teacher data shown in FIG. 9 includes the aforementioned items, as the environment information.

### (4) Operation of Learning Model Generation Device 10

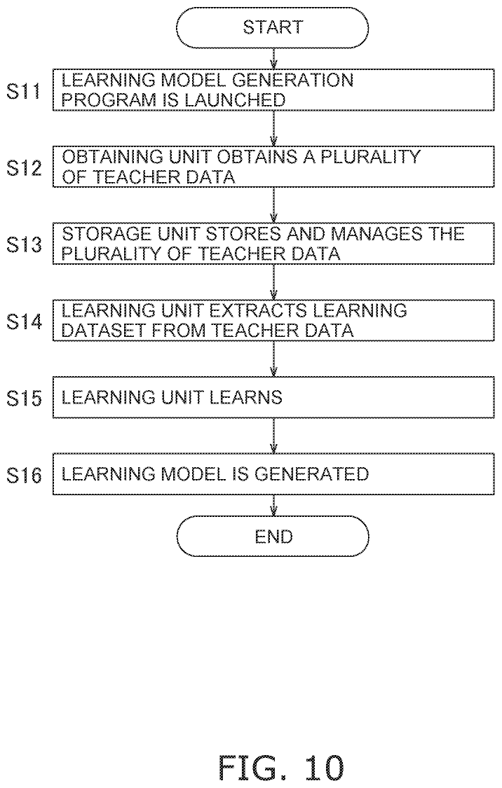

An outline of operation of the learning model generation device 10 is described hereinafter with reference to FIG. 10.

First, in operation S11, the learning model generation device 10 launches the learning model generation program 15 stored in the storage unit 14. The learning model generation device 10 thus operates on the basis of the learning model generation program 15 to start generating a learning model.

In operation S12, the obtaining unit 12 obtains a plurality of teacher data on the basis of the learning model generation program 15.

In operation S13, the obtaining unit 12 stores the plurality of teacher data in the database 16 built in the storage unit 14. The storage unit 14 stores and appropriately manages the plurality of teacher data.

In operation S14, the learning unit 13 extracts a learning dataset from the teacher data stored in the storage unit 14. An A-dataset to be extracted is determined according to a learning objective of the learning model generated by the learning model generation device 10. The dataset is based on the teacher data.

In operation S15, the learning unit 13 learns on the basis of a plurality of datasets thus extracted.

In operation S16, the learning model corresponding to the learning objective is generated on the basis of a result of learning by the learning unit 13 in operation S15.

The operation of the learning model generation device 10 is thus terminated. Note that the sequence, and the like, of the operations of the learning model generation device 10 can be changed accordingly. The learning model thus generated is: implemented to a general-purpose computer or terminal; downloaded as software or an application; or distributed in a state of being stored in a storage medium, for practical application.

### (5) Configuration of the User Device 20

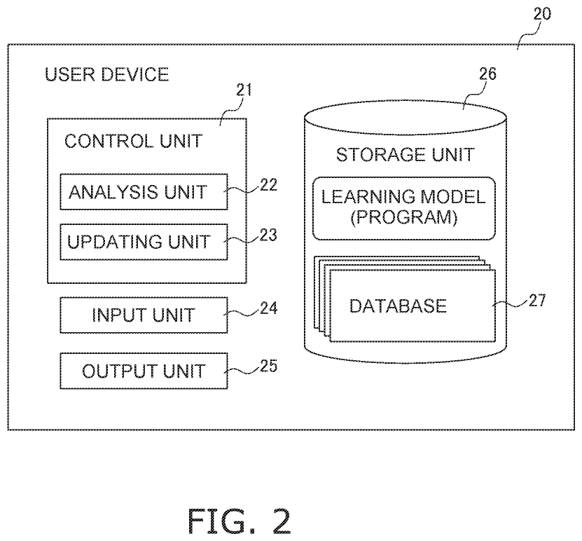

FIG. 2 shows a configuration of the user device 20 used by a user in the present embodiment. As used herein, the term “user” refers to a person who inputs some information to the user device 20 or causes the user device 20 to output some information. The user device 20 uses the learning model generated by the learning model generation device 10.

The user device 20 is a device having a function of a computer. The user device 20 may include a communication interface such as an NIC and a DMA controller, and is configured to communicate with the learning model generation device 10, and the like, through a network. Although the user device 20 shown in FIG. 2 is illustrated as a single device, the user device 20 may be a cloud server or a group of cloud servers implemented in a cloud computing environment. Consequently, as for a hardware configuration, the user device 20 is not required to be accommodated in a single housing or provided as a single device. For example, the user device 20 is configured in such a way that hardware resources thereof are dynamically connected and disconnected according to a load.

The user device 20 includes, for example, an input unit 24, an output unit 25, a control unit 21, and a storage unit 26.

### (5-1) Input Unit 24

The input unit 24 is, for example, a keyboard, a touch screen, a mouse, and the like. The user can input information to the user device 20 through the input unit 24.

### (5-2) Output Unit 25

The output unit 25 is, for example, a display, a printer, and the like. The output unit 25 is capable of outputting a result of analysis by the user device 20 using the learning model as well.

### (5-3) Control Unit 21

The control unit 21 is, for example, a CPU and executes control of an overall operation of the user device 20. The control unit 21 includes function units such as an analysis unit 22, and an updating unit 23.

The analysis unit 22 of the control unit 21 analyzes the input information being input through the input unit 24, by using the learning model as a program stored in the storage unit 26 in advance. The analysis unit 22 employs the aforementioned machine learning approach for analysis; however, the present disclosure is not limited thereto. The analysis unit 22 can output a correct answer even to unknown input information, by using the learning model having learned in the learning model generation device 10.

The updating unit 23 updates the learning model stored in the storage unit 26 to an optimal (or improved) state, in order to obtain a high-quality learning model. The updating unit 23 optimizes weighting between neurons in each layer in a neural network, for example.

### (5-4) Storage Unit 26

The storage unit 26 is an example of the storage medium and may be, for example, a flash memory, a RAM, an HDD, or the like. The storage unit 26 includes the learning model to be executed by the control unit 21, being stored in advance. The storage unit 26 is provided with a database 27 in which a plurality of the teacher data are stored and appropriately managed. Note that, in addition thereto, the storage unit 26 may also store information such as the learning dataset. The teacher data stored in the storage unit 26 is information such as the base material information, the treatment agent information, the evaluation, the environment information as described above.

### (6) Operation of User Device 20

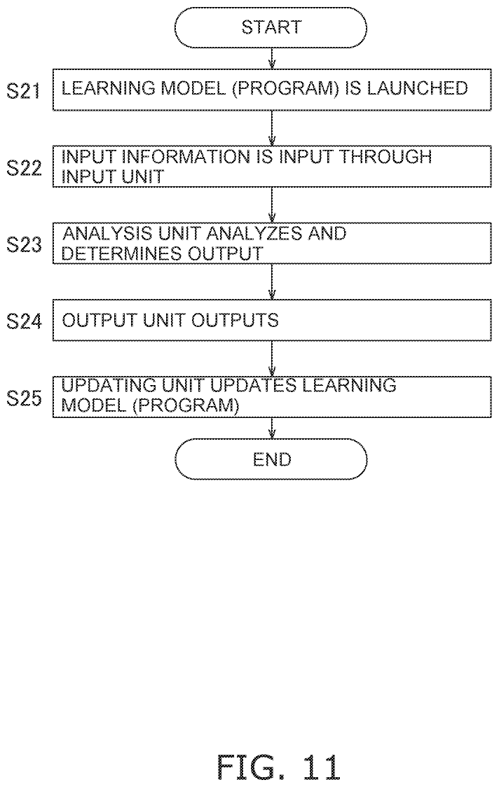

An outline of operation of the user device 20 is described hereinafter with reference to FIG. 11. The user device 20 is in such a state that the learning model generated by the learning model generation device 10 is stored in the storage unit 26.

First, in operation S21, the user device 20 launches the learning model stored in the storage unit 26. The user device 20 operates on the basis of the learning model.

In operation S22, the user who uses the user device 20 inputs input information through the input unit 24. The input information input through the input unit 24 is transmitted to the control unit 21.

In operation S23, the analysis unit 22 of the control unit 21 receives the input information from the input unit 24, analyzes the input information, and determines information to be output from the output unit. The information determined by the analysis unit 22 is transmitted to the output unit 25.

In operation S24, the output unit 25 outputs result information received from the analysis unit 22.

In operation S25, the updating unit 23 updates the learning model to an optimal (or improved) state on the basis of the input information, the result information, and the like.

The operation of the user device 20 is thus terminated. Note that the sequence, and the like, of the operation of the user device 20 can be changed accordingly.

### (7) Specific Examples

Hereinafter, specific examples of using the learning model generation device 10 and the user device 20 described above are explained.

### (7-1) Water-Repellency Learning Model

In this section, a water-repellency learning model that outputs water-repellency is explained.

### (7-1-1) Water-Repellency Learning Model Generation Device 10

In order to generate the water-repellency learning model, the water-repellency learning model generation device 10 may obtain a plurality of teacher data including information regarding at least a type of a base material, a type of a dye with which a surface of the base material is dyed, a type of a monomer constituting a repellent polymer contained in the surface-treating agent, a content of a monomeric unit in the repellent polymer, a content of the repellent polymer in the surface-treating agent, a type of a solvent, a content of the solvent in the surface-treating agent, a type of a surfactant and a content of the surfactant in the surface-treating agent, and water-repellency information. Note that the water-repellency learning model generation device 10 may also obtain other information.

Through learning based on the teacher data thus obtained, the water-repellency learning model generation device 10 can generate the water-repellency learning model that receives as inputs: the base material information including information regarding the type of a base material and the type of a dye with which a surface of the base material is dyed; and the treatment agent information including information regarding the type of a monomer constituting a repellent polymer contained in the surface-treating agent, the content of a monomeric unit in the repellent polymer, the content of the repellent polymer in the surface-treating agent, the type of a solvent, the content of the solvent in the surface-treating agent, and the type of a surfactant and the content of the surfactant in the surface-treating agent, and outputs water-repellency information.

### (7-1-2) User Device 20 Using Water-Repellency Learning Model

The user device 20 is configured to use the water-repellency learning model. The user who uses the user device 20 inputs to the user device 20: the base material information including information regarding the type of a base material and the type of a dye with which a surface of the base material is dyed; and the treatment agent information including information regarding the type of a monomer constituting a repellent polymer contained in the surface-treating agent, the content of a monomeric unit in the repellent polymer, the content of the repellent polymer in the surface-treating agent, the type of a solvent, the content of the solvent in the surface-treating agent, and the type of a surfactant and the content of the surfactant in the surface-treating agent.

The user device 20 uses the water-repellency learning model to determine the water-repellency information. The output unit 25 outputs the water-repellency information thus determined.

### (7-2) Oil-repellency learning model

In this section, an oil-repellency learning model that outputs oil-repellency is explained.

### (7-2-1) Oil-repellency learning model generation device 10

In order to generate the oil-repellency learning model, the oil-repellency learning model generation device 10 may obtain a plurality of teacher data including information regarding at least a type of a base material, a type of a dye with which a surface of the base material is dyed, a type of a monomer constituting a repellent polymer contained in the surface-treating agent, a content of a monomeric unit in the repellent polymer, a content of the repellent polymer in the surface-treating agent, a type of a solvent, a content of the solvent in the surface-treating agent, a type of a surfactant and a content of the surfactant in the surface-treating agent, and oil-repellency information. Note that the oil-repellency learning model generation device 10 may also obtain other information.

Through learning based on the teacher data thus obtained, the oil-repellency learning model generation device 10 can generate the oil-repellency learning model that receives as inputs: the base material information including information regarding the type of a base material and the type of a dye with which a surface of the base material is dyed; and the treatment agent information including information regarding the type of a monomer constituting a repellent polymer contained in the surface-treating agent, the content of a monomeric unit in the repellent polymer, the content of the repellent polymer in the surface-treating agent, the type of a solvent, the content of the solvent in the surface-treating agent, and the type of a surfactant and the content of the surfactant in the surface-treating agent, and outputs oil-repellency information.

### (7-2-2) User device 20 Using Oil-Repellency Learning Model

The user device 20 is configured to use the oil-repellency learning model. The user who uses the user device 20 inputs to the user device 20: the base material information including information regarding the type of a base material and the type of a dye with which a surface of the base material is dyed; and the treatment agent information including information regarding the type of a monomer constituting a repellent polymer contained in the surface-treating agent, the content of a monomeric unit in the repellent polymer, the content of the repellent polymer in the surface-treating agent, the type of a solvent, the content of the solvent in the surface-treating agent, and the type of a surfactant and the content of the surfactant in the surface-treating agent.

The user device 20 uses the oil-repellency learning model to determine the oil-repellency information. The output unit 25 outputs the oil-repellency information thus determined.

### (7-3) Antifouling Property Learning Model

In this section, an antifouling property learning model that outputs antifouling property is explained.

### (7-3-1) Antifouling Property Learning Model Generation Device 10

In order to generate the antifouling property learning model, the antifouling property learning model generation device 10 may obtain a plurality of teacher data including information regarding at least a type of a base material, a type of a dye with which a surface of the base material is dyed, a type of a monomer constituting a repellent polymer contained in the surface-treating agent, a content of a monomeric unit in the repellent polymer, a content of the repellent polymer in the surface-treating agent, a type of a solvent, a content of the solvent in the surface-treating agent, a type of a surfactant and a content of the surfactant in the surface-treating agent, and antifouling property information. Note that the antifouling property learning model generation device 10 may also obtain other information.

Through learning based on the teacher data thus obtained, the antifouling property learning model generation device 10 can generate the antifouling property learning model that receives as inputs: the base material information including information regarding the type of a base material and the type of a dye with which a surface of the base material is dyed; and the treatment agent information including information regarding the type of a monomer constituting a repellent polymer contained in the surface-treating agent, the content of a monomeric unit in the repellent polymer, the content of the repellent polymer in the surface-treating agent, the type of a solvent, the content of the solvent in the surface-treating agent, and the type of a surfactant and the content of the surfactant in the surface-treating agent, and outputs antifouling property information.

### (7-3-2) User Device 20 Using Antifouling Property Learning Model

The user device 20 is configured to use the antifouling property learning model. The user who uses the user device 20 inputs to the user device 20: the base material information including information regarding the type of a base material and the type of a dye with which a surface of the base material is dyed; and the treatment agent information including information regarding the type of a monomer constituting a repellent polymer contained in the surface-treating agent, the content of a monomeric unit in the repellent polymer, the content of the repellent polymer in the surface-treating agent, the type of a solvent, the content of the solvent in the surface-treating agent, and the type of a surfactant and the content of the surfactant in the surface-treating agent.

The user device 20 uses the antifouling property learning model to determine the antifouling property information. The output unit 25 outputs the antifouling property information thus determined.

### (7-4) Processing Stability Learning Model

In this section, a processing stability learning model that outputs processing stability is explained.

### (7-4-1) Processing Stability Learning Model Generation Device 10

In order to generate the processing stability learning model, the processing stability learning model generation device 10 may obtain a plurality of teacher data including information regarding at least a type of a base material, a type of a dye with which a surface of the base material is dyed, a type of a monomer constituting a repellent polymer contained in the surface-treating agent, a content of a monomeric unit in the repellent polymer, a content of the repellent polymer in the surface-treating agent, a type of a solvent, a content of the solvent in the surface-treating agent, a type of a surfactant and a content of the surfactant in the surface-treating agent, and processing stability information. Note that the processing stability learning model generation device 10 may also obtain other information.

Through learning based on the teacher data thus obtained, the processing stability learning model generation device 10 can generate the processing stability learning model that receives as inputs: the base material information including information regarding the type of a base material and the type of a dye with which a surface of the base material is dyed; and the treatment agent information including information regarding the type of a monomer constituting a repellent polymer contained in the surface-treating agent, the content of a monomeric unit in the repellent polymer, the content of the repellent polymer in the surface-treating agent, the type of a solvent, the content of the solvent in the surface-treating agent, and the type of a surfactant and the content of the surfactant in the surface-treating agent, and outputs processing stability information.

### (7-4-2) User Device 20 Using Processing Stability Learning Model

The user device 20 is configured to use the processing stability learning model. The user who uses the user device 20 inputs to the user device 20: the base material information including information regarding the type of a base material and the type of a dye with which a surface of the base material is dyed; and the treatment agent information including information regarding the type of a monomer constituting a repellent polymer contained in the surface-treating agent, the content of a monomeric unit in the repellent polymer, the content of the repellent polymer in the surface-treating agent, the type of a solvent, the content of the solvent in the surface-treating agent, and the type of a surfactant and the content of the surfactant in the surface-treating agent.

The user device 20 uses the processing stability learning model to determine the processing stability information. The output unit 25 outputs the processing stability information thus determined.

### (7-5) Water-Repellent Agent Learning Model

In this section, a water-repellent agent learning model that outputs the optimal (or improved) water-repellent agent is explained.

### (7-5-1) Water-Repellent Agent Learning Model Generation Device 10

In order to generate the water-repellent agent learning model, the water-repellent agent learning model generation device 10 may obtain a plurality of teacher data including information regarding at least a type of a base material, a type of a dye with which a surface of the base material is dyed, a type of a monomer constituting a repellent polymer contained in the surface-treating agent, a content of a monomeric unit in the repellent polymer, a content of the repellent polymer in the surface-treating agent, a type of a solvent, a content of the solvent in the surface-treating agent, a type of a surfactant and a content of the surfactant in the surface-treating agent, and water-repellency information. Note that the water-repellent agent learning model generation device 10 may also obtain other information.

Through learning based on the teacher data thus obtained, the water-repellent agent learning model generation device 10 can generate the water-repellent agent learning model that receives as an input the base material information including information regarding the type of a base material and the type of a dye with which a surface of the base material is dyed, and outputs repellent agent information that is optimal (or improved) for the base material.

### (7-5-2) User Device 20 Using Water-Repellent Agent Learning Model

The user device 20 is configured to use the water-repellent agent learning model. The user who uses the user device 20 inputs to the user device 20 the base material information including information regarding the type of a base material and the type of a dye with which a surface of the base material is dyed.

The user device 20 uses the water-repellent agent learning model to determine the repellent agent information that is optimal (or improved) for the base material. The output unit 25 outputs the repellent agent information thus determined.

### (7-6) Oil-Repellent Agent Learning Model

In this section, an oil-repellent agent learning model that outputs the optimal (or improved) oil-repellent agent is explained.

### (7-6-1) Oil-Repellent Agent Learning Model Generation Device 10

In order to generate the oil-repellent agent learning model, the oil-repellent agent learning model generation device 10 may obtain a plurality of teacher data including information regarding at least a type of a base material, a type of a dye with which a surface of the base material is dyed, oil-repellency information, a type of a monomer constituting a repellent polymer contained in the surface-treating agent, a content of a monomeric unit in the repellent polymer, a content of the repellent polymer in the surface-treating agent, a type of a solvent, a content of the solvent in the surface-treating agent, a type of a surfactant and a content of the surfactant in the surface-treating agent, and oil-repellency information. Note that the oil-repellency learning model generation device 10 may also obtain other information.

Through learning based on the teacher data thus obtained, the oil-repellent agent learning model generation device 10 can generate the oil-repellent agent learning model that receives as an input the base material information including information regarding the type of a base material and the type of a dye with which a surface of the base material is dyed, and outputs repellent agent information that is optimal (or improved) for the base material.

### (7-6-2) User Device 20 Using Oil-Repellent Agent Learning Model

The user device 20 is configured to use the oil-repellent agent learning model. The user who uses the user device 20 inputs to the user device 20 the base material information including information regarding the type of a base material and the type of a dye with which a surface of the base material is dyed.

The user device 20 uses the oil-repellent agent learning model to determine the repellent agent information that is optimal (or improved) for the base material. The output unit 25 outputs the repellent agent information thus determined.

### (8) Characteristic Features

### (8-1)

A learning model generation method according to the present embodiment generates a learning model for determining by using a computer an evaluation of an article in which a surface-treating agent is fixed onto a base material. The learning model generation method includes the obtaining operation S12, the learning operation S15, and the generating operation S16. In the obtaining operation S12, the computer obtains teacher data. The teacher data includes base material information, treatment agent information, and an evaluation of an article. The base material information is information regarding a base material. The treatment agent information is information regarding a surface-treating agent. In the learning operation S15, the computer learns on the basis of a plurality of the teacher data obtained in the obtaining operation S12. In the generating operation S16, the computer generates the learning model on the basis of a result of learning in the learning operation S15. The article is obtained by fixing the surface-treating agent onto the base material. The learning model receives input information as an input, and outputs the evaluation. The input information is unknown information different from the teacher data. The input information includes at least the base material information and the treatment agent information.

The computer uses a learning model, as a program, having further learned the base material information, the treatment agent information, and the evaluation as the teacher data as described above, to determine an evaluation. The learning model includes the input operation S22, the determination operation S23, and the output operation S24. In the input operation S22, unknown information different from the teacher data, including the base material information and the treatment agent information, is input. In the determination operation S23, the computer uses the learning model to determine the evaluation. In the output operation S24, the computer outputs the evaluation determined in the determination operation S23.

Conventionally, an article in which a surface-treating agent is fixed to a base material has been evaluated on site by testing every combination of various base materials and surface-treating agents. Such a conventional evaluation method requires extensive time and a considerable number of operations, and there has been a demand for an improved evaluation method.

In addition, as disclosed in Patent Literature 2 (JPB No. 4393595), programs and the like, employing neural networks have been designed for outputting an optimal combination in other fields; however, in the special field of a water-repellent agent, no programs, or the like, employing neural networks have been designed.

The learning model generated by the learning model generation method according to the present embodiment enables evaluation by using a computer. Reduction of the extensive time and the considerable number of operations, which have been conventionally required, is thus enabled. The reduction of the number of operations in turn enables reduction of human resources and cost for the evaluation.

### (8-2)

A learning model generation method according to the present embodiment generates a learning model for determining, by using a computer, an optimal (or improved) surface-treating agent for a base material. The learning model generation method includes the obtaining operation S12, the learning operation S15, and the generating operation S16. In the obtaining operation S12, the computer obtains teacher data. The teacher data includes base material information, treatment agent information, and an evaluation. The base material information is information regarding a base material. The treatment agent information is information regarding a surface-treating agent. The evaluation is regarding the article in which the surface-treating agent is fixed onto the base material. In the learning operation S15, the computer learns on the basis of a plurality of the teacher data obtained in the obtaining operation S12. In the generating operation S16, the computer generates the learning model on the basis of a result of learning in the learning operation S15. The article is obtained by fixing the surface-treating agent onto the base material. The learning model receives input information as an input, and outputs the evaluation. The input information is unknown information different from the teacher data. The input information includes at least the base material information.

The computer uses a learning model, as a program, having further learned the base material information, the treatment agent information, and the evaluation as the teacher data as described above, to determine treatment agent information. The program includes the input operation S22, the determination operation S23, and the output operation S24. In the input operation S22, unknown information different from the teacher data, including the base material information, is input. In the determination operation S23, the computer uses the learning model to determine treatment agent information that is optimal (or improved) for the base material. In the output operation S24, the computer outputs the treatment agent information determined in the determination operation S23.

With the conventional evaluation method, when a poorly-evaluated combination of a base material and a surface-treating agent is found on site, the combination may need research and improvement in a research institution, whereby selection of a surface-treating agent optimal (or improved) for a substrate requires extensive time and a considerable number of operations.

The learning model generated by the learning model generation method according to the present embodiment enables determination of an optimal (or improved) surface-treating agent for a base material by using a computer. Time, the number of operations, human resources, cost, and the like, for selecting an optimal (or improved) surface-treating agent can thus be reduced.

### (8-3)

In the learning operation S15 of the learning model generation method according to the present embodiment, the learning is preferably performed by a regression analysis and/or ensemble learning that is a combination of a plurality of regression analyses.

The evaluation by the learning model as a program according to the present embodiment is any of water-repellency information, oil-repellency information, antifouling property information, or processing stability information. The water-repellency information is information regarding water-repellency of the article. The oil-repellency information is information regarding oil-repellency of the article. The antifouling property information is information regarding an antifouling property of the article. The processing stability information is preferably information regarding processing stability of the article.

The base material is preferably a textile product.

The base material information includes information regarding at least a type of the textile product and a type of a dye. The treatment agent information includes information regarding at least a type of a monomer constituting a repellent polymer contained in the surface-treating agent, a content of a monomeric unit in the polymer, a content of the repellent polymer in the surface-treating agent, a type of a solvent and a content of the solvent in the surface-treating agent, and a type of a surfactant and a content of the surfactant in the surface-treating agent.

The teacher data includes environment information during processing of the base material. The environment information includes information regarding any of temperature, humidity, curing temperature, or processing speed during the processing of the base material. The base material information preferably further includes information regarding any of a color, a weave, basis weight, yarn thickness, or zeta potential of the textile product. The treatment agent information further includes information regarding any item of: a type and a content of an additive to be added to the surface-treating agent; pH of the surface-treating agent; or zeta potential thereof.

The teacher data preferably includes information regarding many items, and the greater number of pieces as possible of the teacher data is preferred. A more accurate output can thus be obtained.

### (8-4)

The learning model as a program according to the present embodiment may also be distributed in a form of a storage medium storing the program.

### (8-5)

The learning model according to the present embodiment is a learned model having learned by the learning model generation method. The learned model causes a computer to function to: perform calculation based on a weighting coefficient of a neural network with respect to base material information, which is information regarding the base material, and treatment agent information, which is information regarding a surface-treating agent to be fixed onto the base material, being input to an input layer of the neural network; and output water-repellency information or oil-repellency information of an article from an output layer of the neural network. The weighting coefficient is obtained through learning of at least the base material information, the treatment agent information, and an evaluation of the base material in which the surface-treating agent is fixed onto the base material, as teacher data. The article is obtained by fixing the surface-treating agent onto the base material.

### (8-6)

The learned model causes a computer to function to: perform calculation based on a weighting coefficient of a neural network with respect to base material information, which is information regarding the base material, being input to an input layer of the neural network; and to output treatment agent information that is optimal (or improved) for the base material from an output layer of the neural network. The weighting coefficient is obtained through learning of at least the base material information, the treatment agent information, and an evaluation of the base material onto which the surface-treating agent is fixed, as teacher data. The treatment agent information is information regarding a surface-treating agent to be fixed onto the base material. The article is obtained by fixing the surface-treating agent onto the base material.

### (9)

The embodiment of the present disclosure has been described in the foregoing; however, it should be construed that various modifications of modes and details can be made without departing from the spirit and scope of the present disclosure set forth in Claims.

### REFERENCE SIGNS LIST

S12 Obtaining operation
S15 Learning operation
S16 Generating operation
S22 Input operation
S23 Determination operation
S24 Output operation

### CITATION LIST

### Patent Literature

[Patent Literature 1] JPA No. 2018-535281
[Patent Literature 2] JPB No. 4393595

1. A learning model generation method of generating a learning model for determining, by a processor, a first evaluation of a first article in which a first surface-treating agent is fixed onto a first base material, the learning model generation method comprising:
obtaining, by the processor, as teacher data, information including at least second base material information regarding a second base material, second treatment agent information regarding a second surface-treating agent, and a second evaluation of a second article;
learning, by the processor, based on the teacher data; and
generating, by the processor, the learning model based on the learning,
wherein:
the first article is obtained by fixing the first surface-treating agent onto the first base material:
the second article is obtained by fixing the second surface-treating agent onto the second base material;
the learning model is configured to receive input information, which is different from the teacher data, as an input, and output the first evaluation of the first article; and
the input information includes at least the first base material information regarding the first base material, and first treatment agent information regarding the first surface-treating agent.

2. A learning model generation method comprising:
obtaining, by a processor, as teacher data, information including at least first base material information regarding a first base material, first treatment agent information regarding a first surface-treating agent to be fixed onto a first base material, and a first evaluation of a first article in which the first surface-treating agent is fixed onto the first base material;
learning, by the processor, based on the teacher data; and
generating, by the processor, a learning model based on the learning,
wherein:
the first article is obtained by fixing the first surface-treating agent onto the first base material;
a second article is obtained by fixing a second surface-treating agent onto a second base material.
the learning model is configured to receive input information, which is different from the teacher data, as an input, and output second treatment agent information for the second base material; and
the input information includes at least the second base material information regarding the second base material, and information regarding the a second evaluation of the second base material.

3. The learning model generation method as claimed in claim 1, wherein the learning is performed by a regression analysis or ensemble learning that is a combination of a plurality of regression analyses.

4. A device for determining, by using a learning model, a first evaluation of a first article in which a first surface treating agent is fixed onto a first base material, the device comprising:
a memory configured to store a program; and
a processor configured to execute the program to:
receive input information as an input;
determine, using the input information and the learning model, the first evaluation of the first article in which the first surface-treating agent is fixed onto the first base material; and
output the first evaluation,
wherein:
the first article is obtained by fixing the first surface-treating agent onto the first base material;
a second article is obtained by fixing a second surface-treating agent onto a second base material;
the learning model is configured to learn using teacher data including information including at least second base material information regarding the second base material, second treatment agent information regarding the second surface-treating agent, and a second evaluation of the second article; and
the input information is different from the teacher data, and includes at least the first base material information and the first treatment agent information.

5. A device for determining, using a learning model, first treatment agent information regarding a first surface-treatment agent to be fixed onto a first base material of a first article, the device comprising:
a memory configured to store a program; and
a processor configured to execute the program to:
receive input information as an input;
determine, using the input information and the learning model, the first treatment agent information; and
output the first treatment agent information,
wherein:
the learning model is configured to learn using teacher data including information including at least second base material information regarding a second base material, second treatment agent information regarding a second surface-treating agent to be fixed onto the second base material, and a second evaluation of a second article in which the second surface-treating agent is fixed onto the second base material;
the input information is different from the teacher data, and includes at least the first base material information and information regarding a first evaluation of the first article;
the first article is obtained by fixing the first surface-treating agent onto the first base material; and
the second article is obtained by fixing the second surface-treating agent onto the second base material.

6. The device as claimed in claim 4, wherein the first evaluation includes at least one of water-repellency information regarding water-repellency of the first article, oil-repellency information regarding oil-repellency of the first article, antifouling property information regarding an antifouling property of the first article or processing stability information regarding processing stability of the first article.

7. The device as claimed in claim 4, wherein the first base material is a textile product.

8. The device as claimed in claim 7, wherein:
the first base material information comprises information regarding at least a type of the textile product and a type of a dye; and
the first treatment agent information comprises information regarding at least a type of a monomer constituting a repellent polymer contained in the first surface-treating agent, a content of the monomer in the repellent polymer, a content of the repellent polymer in the surface-treating agent, a type of a solvent and a content of the solvent in the first surface-treating agent, and a type of a surfactant and a content of the surfactant in the first surface-treating agent.

9. The device as claimed in claim 8, wherein:
the teacher data further comprises environment information regarding an environment during processing of the second base material;
the environment information comprises information regarding at least one of a concentration of the second surface-treating agent in a treatment tank, a temperature of the environment, a humidity of the environment, a curing temperature, or a processing speed during the processing of the second base material;
the second base material information further comprises information regarding at least one of a color, a weave, a basis weight, a yarn thickness, or a zeta potential of a second textile product; and
the second treatment agent information further comprises information regarding at least one of a type and a content of an additive to be added to the second surface-treating agent, a pH of the second surface-treating agent, or a zeta potential of the second-surface treating agent.

10. A non-transitory computer-readable medium storing a program for determining, by using a learning model, a first evaluation of a first article in which a first surface treating agent is fixed onto a first base material, the program being configured to cause a processor to:
receive input information as an input
determine, using the input information and the learning model, the first evaluation of the first article in which the first surface-treating agent is fixed onto the first base material; and
output the first evaluation,
wherein:
the first article is obtained by fixing the first surface-treating agent onto the first base material;
a second article is obtained by fixing a second surface-treating agent onto a second base material;
the learning model is configured to learn using teacher data including information including at least second base material information regarding the second base material, second treatment agent information regarding the second surface-treating agent, and a second evaluation of the second article; and
the input information is different from the teacher data, and includes at least the second base material information and the second treatment agent information.

11. A device comprising:
a memory configured to store a learned model; and
a processor configured to, using the learned model, perform calculation based on a weighting coefficient of a neural network with respect to first base material information regarding a first base material and first treatment agent information regarding a first surface-treating agent being input to an input layer of the neural network, and output a first evaluation of a first article from an output layer of the neural network, wherein:
the weighting coefficient is obtained through learning of the learned model using at least second base material information, second treatment agent information, and a second evaluation as teacher data;
the second base material information is information regarding a second base material;
the second treatment agent information is information regarding a second surface-treating agent to be fixed onto the second base material;
the second evaluation is regarding the second article in which the second surface-treating agent is fixed onto the second base material;
the first article is obtained by fixing the first surface-treating agent onto the first base material; and
the second article is obtained by fixing the second surface-treating agent onto the second base material.

12. A device comprising:
a memory configured to store a learned model; and
a processor configured to, using the learned model, perform calculation based on a weighting coefficient of a neural network with respect to first base material information regarding a first material and information regarding a first evaluation being input to an input layer of the neural network, and output first treatment agent information regarding a first surface-treating agent to be fixed onto the first base material from an output layer of the neural network, wherein:
the weighting coefficient is obtained through learning of the learned model using at least second base material information, second treatment agent information, and a second evaluation as teacher data;
the second base material information is information regarding the second base material;
the second treatment agent information is information regarding a second surface-treating agent to be fixed onto the second base material;
the second evaluation is regarding a second article in which the second surface-treating agent is fixed onto the second base material;
the first article is obtained by fixing the first surface-treating agent onto the first base material; and
the second article is obtained by fixing the second surface-treating agent onto the second base material.
